(19) United States
(12) Patent Application Publication
(10) Pub. No.: US 2008/0177171 A1
(43) Pub. Date: Jul. 24, 2008

(54) ELECTRICAL PATCH PANEL FOR ISOLATION ENVIRONMENTS

(75) Inventors: James E. FRANCESANGELI, Hinckley, OH (US); Leonard J. CIKOTTE, Garrettsville, OH (US); Eugene A. FATICA, Highland Heights, OH (US); Robert C. GAUSS, Aurora, OH (US)

Correspondence Address: PHILIPS INTELLECTUAL PROPERTY & STANDARDS, 595 MINER ROAD, CLEVELAND, OH, 44143, US

(73) Assignee: KONINKLIJKE PHILIPS ELECTRONICS N.V., Eindhoven (NL)

(21) Appl. No.: 11/844,580

(22) Filed: Aug. 24, 2007

Related U.S. Application Data
(63) Continuation of application No. PCT/US07/69836, filed on May 29, 2007.
(60) Provisional application No. 60/804,308, filed on Jun. 9, 2006.

(51) Int. Cl. A61G 10/00; H01R 13/66; A61B 6/03; A61B 5/055

(52) U.S. Cl. 600/410; 439/540.1; 378/20; 600/21

(57) ABSTRACT

A through-hole panel is mounted on a barrier between a hot zone maintained at a selected isolation level and a cold zone not maintained at the selected isolation level. Hermetically sealed electrical feedthroughs each include a housing and cold- and hot-side electrical receptacles, and are hermetically sealed into through-holes of the through-hole panel with the cold- and hot-side electrical receptacles extending into the respective cold and hot zones. A surface of the through-hole panel and a portion of the feedthroughs exposed to the hot zone are substantially resistant to corrosive decontamination agents used in the hot zone. A medical imaging instrument in the cold zone images an interior volume of a generally tubular imaging window that is in communication with the hot zone and is isolated from the cold zone. An auxiliary instrument in the hot zone operatively electrically communicates with the medical imaging instrument via the feedthroughs.

### CROSS REFERENCE TO RELATED APPLICATIONS

This application is a Continuation of PCT/U.S.07/69836 filed May 29, 2007 which claims the benefit of U.S. provisional application Ser. No. 60/804,308 filed Jun. 9, 2006, the subject of which is incorporated herein by reference.

This invention was made with Government support under grant no. N01-A0-60001 awarded by the National Institutes of Health (NIH). The Government has certain rights in this invention.

### BACKGROUND

The following relates to the environmental isolation and safety arts, and is described by way of example with reference to medical imaging systems for imaging infectious subjects in contained environments configured to isolate the biological contagion. The following finds more general application in isolation environments for researching, processing, or otherwise manipulating or containing radioactive, toxic, biologically infectious, or other hazardous substances, subjects, objects, or so forth. Conversely, it also finds application in conjunction with isolated environments such as clean rooms, sterile rooms, inert gas environments, and so forth, that are controlled to limit contamination from normal environmental conditions.

Biologically hazardous and highly contagious diseases are an increasing public health concern. Increasing air travel promotes the rapid worldwide spread of contagions. Bioterrorism is another potential route to public exposure to hazardous contagions. Effective response to an outbreak of a contagion is facilitated by knowledge of the infectious agent (that is, the type or species of virus, bacterium, prion, or so forth), effect of counteragents (such as drugs or other types of treatment), transmission pathways (such as airborne transmission, contact transmission, or so forth), incubation period before symptoms arise, and so forth. This knowledge is gained by suitable laboratory studies, which must be conducted in a suitably biologically isolated environment.

The National Institute of Health (NIH) and Center for Disease Control (CDC) have promulgated operational criteria for laboratories conducting biological research into hazardous contagions. Four levels of isolation have been defined: BioSafety Level 1 (BSL-1), BSL-2, BSL-3, and BSL-4, with the level of isolation increasing with increasing BSL level. The BSL-3 level requires isolation steps such as physical separation of the laboratory working area from access corridors and controlled air flow. BSL-4 requires an isolated laboratory space (sometimes called the “hot zone”) with dedicated air flow. The hot zone is a room, room partition, or building that is sealed from the environment to prevent escape of airborne contagions, and laboratory personnel working within the hot zone wear sealed environmental suits with self-contained breathing apparatuses. Laboratory personnel and any items that leave the hot zone must undergo specified decontamination procedures before being admitted to a “cold zone” outside the BSL-4 environment. The surfaces in the BSL-4 hot zone should also be resistant to the types of corrosive cleaners typically used in biological decontamination, such as Clydox-S, Microchem, Quat TB, Para-Formaldehyde, Chlorine-Dioxide, Vaporized Hydrogen Peroxide, Ammonium Carbonate, and so forth. Other factors in design of the BSL-4 environment include minimizing or eliminating fine operational features (such as small fasteners, control buttons, or the like which are difficult to manipulate while wearing hazardous material, i.e. HASMAT, suits or other isolation suits with gloves), eliminating sharp edges, corners, or rough features that can tear, puncture, cut, or otherwise rupture isolation suits, and providing a high level of redundancy or backup for systems and components in the hot zone.

These considerations for BSL-4 environments are also applicable to other isolation environments, such as clean rooms, sterile rooms, inert gas environments, and so forth, that are controlled to limit contamination from normal environmental conditions. For example, it may be advantageous to perform drug development experiments in a sterile zone to avoid inadvertent infection of the test subject animals.

To provide a functional isolation zone, various consumables such as water, electricity, air, or so forth must pass into and/or out of one or more barriers that seal the isolation zone. Typically, the barrier is a wall of a suitably biologically impermeable, corrosion resistant material such as stainless steel, steel coated with stainless steel or Teflon, or so forth. The barrier should be amenable to decontamination using corrosive chemicals. In isolation existing BSL-4 environments, for example, electrical feedthrough wires are typically potted into the barrier. For example, a typical approach for an electrical wire is to drill an opening in the barrier at the point where the electrical wire is to pass into the hot zone, strip insulation off the portion of the electrical wire to be potted, and pot the stripped wire portion into the drilled opening of the barrier. Stripping of the wire before potting advantageously promotes a good seal and eliminates potential leakage paths through the insulation, or at the interface between the insulation and the wire, or at the interface between the insulation and the potting material.

Potting electrical wires into the barrier has known disadvantages. Potting is labor-intensive and results in a permanently installed electrical wire. Subsequent re-wiring would require breaking containment of the isolation zone before breaking the potted seal. In the case of a BSL-4 isolation zone, the continuous length of electrical wire that passes through the barrier includes a portion in the cold zone with insulation that is resistant to the corrosive decontamination chemicals used on the hot side, even though the wire portion in the cold zone is not decontaminated. These disadvantages multiply as the number of electrical wires passing through the barrier increases. However, the potting approach continues to be used in BSL-4 and other isolation environments.

The present application provides new and improved electrical patch panels for use in isolation environments, such as biological isolation environments (e.g., BSL-3 and BSL-4 environments), nuclear isolation environments, toxic isolation environments, ambient atmosphere isolation environments, and so forth, which overcome the above-referenced problems and others.

### SUMMARY

In accordance with one aspect, an electrical patch panel is disclosed for use in communicating electrical power or electrical signals across a barrier between an isolation zone and an ambient zone. A through-hole panel is mounted on the barrier between the isolation zone and the ambient zone. A plurality of electrical feedthroughs each include a housing disposed in a through-hole of the through-hole panel, an ambient-side electrical receptacle exposed to the ambient zone, an isolation-side electrical receptacle exposed to the isolation zone and electrically connected with the ambient-side electrical receptacle, and potting material disposed in the housing that isolates the isolation-side electrical receptacle from the ambient-side electrical receptacle. An interface or gap between an edge of the through-hole and the electrical feedthrough is sealed such that a pressure differential can be maintained between the isolation and ambient zones.

In accordance with another aspect, a medical imaging system is disclosed. A medical imaging instrument is disposed in a cold zone and arranged to image a subject disposed in a hot zone. At least one electrical feedthrough includes including a housing sealed in a barrier between the hot zone and the cold zone, a cold-side electrical receptacle accessible from the cold zone, and a hot-side electrical receptacle accessible from the hot zone. The medical imaging instrument is electrically accessible from the hot zone via the at least one electrical feedthrough.

In accordance with another aspect, a biological isolation system is disclosed. A hot zone is maintained at a selected level of biological isolation. A through-hole panel is mounted on a barrier between the hot zone and a cold zone that is not maintained at the selected level of biological isolation. A plurality of hermetically sealed electrical feedthroughs are provided, each including a housing, a cold-side electrical receptacle, and a hot-side electrical receptacle. The hermetically sealed electrical feedthroughs are hermetically sealed into through-holes of the through-hole panel with the hot-side electrical receptacle extending into the hot zone and the cold-side electrical receptacle extending into the cold zone. A surface of the through-hole panel exposed to the hot zone and a portion of the hermetically sealed electrical feedthroughs exposed to the hot zone are substantially resistant to one or more corrosive biological decontamination agents used in decontamination of the hot zone.

In accordance with another aspect, a method of providing electrical connections across a barrier of an isolation zone is disclosed. An opening is formed in a barrier of an isolation zone. A sealed electrical feedthrough is inserted at the opening in the barrier. An interface or gap between a housing of the sealed electrical feedthrough and an edge of the barrier is sealed.

In accordance with another aspect, a biological containment environment for imaging is disclosed. An isolation zone is maintained at a selected level of biological isolation. A medical imaging instrument is disposed outside the isolation zone. A tube extends from the isolation zone into an imaging region of the medical imaging instrument via which a subject in the isolation zone can be introduced into the imaging region without breaking containment of the isolation zone. A plurality of hermetically sealed electrical feedthroughs pass through a barrier delimiting the isolation zone. Each hermetically sealed electrical feedthrough includes a hermetically sealed housing with a cold-side electrical receptacle accessible from outside the isolation zone and a hot-side electrical receptacle accessible from within the isolation zone. The hermetically sealed electrical feedthroughs provide electrical communication between the isolation zone and the medical imaging instrument.

One advantage resides in enabling reconfiguration of electrical connections into and out of an isolation environment without breaking containment.

Another advantage resides in providing redundancy in electrical connections into and out of an isolation environment without breaking containment.

Another advantage resides in more efficient construction of electrical connections into and out of an isolation environment.

Still further advantages of the present invention will be appreciated to those of ordinary skill in the art upon reading and understand the following detailed description.

### DRAWINGS

The invention may take form in various components and arrangements of components, and in various steps and arrangements of steps. The drawings are only for purposes of illustrating the preferred embodiments and are not to be construed as limiting the invention.

FIG. 1 shows a diagrammatic perspective view of an isolation facility including a hot zone maintained at the BSL-4 isolation level adjacent a cold zone containing two medical imaging instruments configured to image a subject in the hot zone.

FIG. 2 shows a diagrammatic view of the isolation facility of FIG. 1, with the subject table extended into a first one of the medical imaging instruments.

FIG. 3 diagrammatically shows a view from the hot zone of the patch panel of FIGS. 1 and 2.

FIG. 4 diagrammatically shows a side-sectional view of the through-hole plate of the patch panel mounted on the barrier between the hot zone and the cold zone.

FIG. 5 diagrammatically shows an exploded side-sectional view of one of the electrical feedthroughs of the patch panel.

FIG. 6 diagrammatically shows a side-sectional view of one of the electrical feedthroughs of the patch panel, along with the hot side cable and cold side cable in position to mate with the electrical feedthrough.

### DESCRIPTION

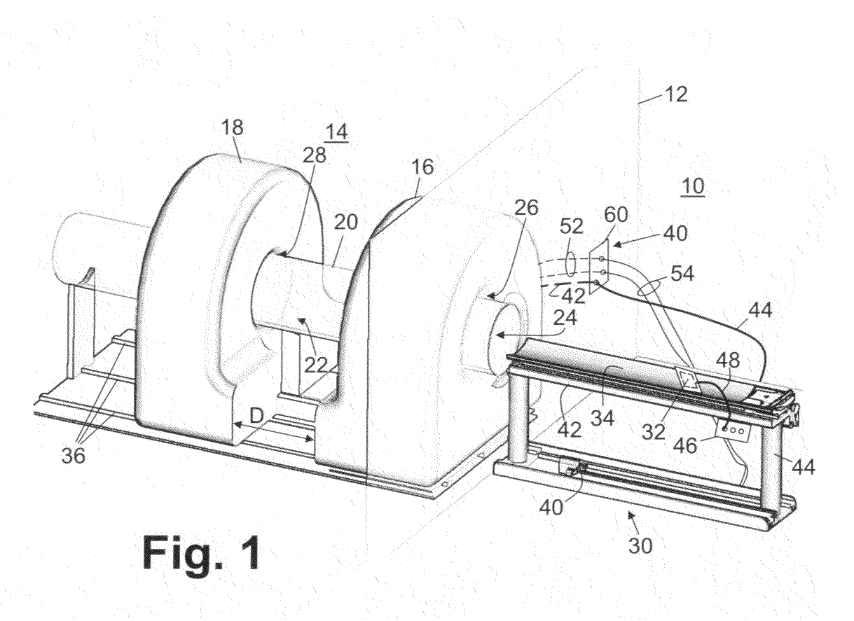

With reference to FIG. 1, an isolation facility includes a hot or isolation zone 10 isolated by a barrier 12 from a cold or ambient zone 14. Although a single representative barrier 12 is shown, typically the hot zone 10 will be enclosed or sealed by a plurality of such barriers, for example by four walls, a floor, and a ceiling defining a sealed room. Access is provided through an airlock door system (not shown). Moreover, while the representative barrier 12 is shown as a transparent barrier, the barrier may be transparent, translucent, or opaque. For example, in some embodiments the hot zone 10 is enclosed by stainless steel walls, floor, and ceiling. The hot zone 10 contains, or may contain, a contagion or infectious agent such as a communicable virus, bacterium, prion, spore, or so forth, or contains or may contain another hazard such as a nerve gas or other toxic chemical, a radioactive material, or so forth. The contagion may be communicable by air, by physical contact, by ingestion, by exchange of bodily fluids, or so forth. The contagion may actually be present in the air or on surfaces within the hot zone 10, or the contagion may be contained within a glovebox or other containment device. In the former case, the hot zone 10 provides primary containment of the contagion; in the latter case, the hot zone 10 provides a backup or failsafe containment for the contagion in the event that it should escape the glovebox or other primary containment. While the hot zone 10 is a biologically contaminated or potentially biologically contaminated hot zone, in other embodiments the hot zone may be a radioactive or potentially radioactive hot zone, a chemically contaminated or potentially chemically contaminated hot zone, or so forth.

In view of the actual or possible presence of the contagion in the hot zone 10, suitable biological safety standards are employed. In some embodiments, the hot zone 10 is maintained at BioSafety Level 4 (BSL-4), which entails such precautions as hermetically sealing off the hot zone 10, keeping the hot zone 10 at a negative differential pressure respective to the cold zone 14, periodically decontaminating the hot zone 10, limiting access to the hot zone 10 to qualified personnel wearing sealed environmental suits with self-contained breathing apparatuses, limiting or eliminating sharp objects or corners in the hot zone 10 (to avoid inadvertent puncturing of the sealed environmental suits), employing a suitable decontamination protocol for personnel or objects leaving the hot zone 10, and so forth. In other embodiments, the safety standards employed in the hot zone are selected based on the type of contagion, radioactive substance, toxic substance, or so forth which is present, or potentially present, in the hot zone.

The isolation facility of FIG. 1 includes one or more medical imaging instruments 16, 18 disposed in the cold zone 14 and configured to image a subject, such as a laboratory test animal, an infected person, a contagion transmission vector such as a plant that may carry the contagion, or so forth, disposed on the hot zone 10. The one or more medical imaging instruments 16, 18 may include, for example, a magnetic resonance (MR) scanner, a positron emission tomography (PET) scanner, a gamma camera for acquiring single-photon emission computed tomography (SPECT) data, a transmission computed tomography (CT) scanner, an x-ray imager, or so forth. Such medical imaging instruments 16, 18 are typically expensive and typically include a large number of parts, some of which may be incompatible with corrosive substances used in decontamination of the hot zone 10.

Accordingly, the medical imaging instruments 16, 18 are disposed in the cold zone 14 and image the subject disposed in the hot zone 10 through a suitable imaging window or tube 20 arranged at the barrier 12 isolating the hot zone 10 from the cold zone 14. In the illustrated embodiment, the imaging window 20 is generally hollow and extends into the cold zone 14 to define an interior volume 22 having an opening 24 communicating with the hot zone 10. The interior volume 22 of the generally hollow imaging window 20 is isolated from the cold zone 14, for example by having the edges of the opening 24 hermetically sealed with the barrier 12 and having a sealed cap or other closure at is far end, which closure may be made of the same material, and is optionally contiguous with the tube. In the illustrated embodiment, the generally hollow imaging window 20 has the shape of a cylinder and passes through a bore 24 of the first medical imaging instrument 16 and through a bore 26 of the second medical imaging instrument 18. It will be appreciated that the illustrated cylindrical generally hollow imaging window 20 is an example—in other contemplated embodiments, the imaging window may be generally hollow with a conical shape having a taper, or may have a circular, elliptical, square, rectangular, or otherwise-shaped cross-section, or the imaging window may be planar (suitable, for example, to enable a medical imaging instrument in the form of a camera to photograph the subject disposed in the hot zone 10), or so forth.

The imaging window 20 allows for the subject in the hot zone 10 to be imaged by the medical imaging instrument 16, 18 disposed in the cold zone 14. Depending upon the imaging modality, the imaging window 20 may or may not be optically transparent. For example, in the case of an MR scanner, the imaging window 20 can be optically opaque or transparent, but should be non-magnetic to enable the radio frequency fields and applied magnetic fields and magnetic field gradients to pass through the imaging window 20 substantially unimpeded. For computed tomography imaging, the imaging window 20 should be made of a material that is substantially transparent to the transmitted x-rays. For PET or SPECT imaging, the imaging window 20 should be made of a material that is substantially transparent to the emitted gamma rays or other radiation emitted by a radiopharmaceutical that is administered to the subject. For photographic imaging, the imaging window 20 should be optically transparent.

Advantageously, the medical imaging instruments 16, 18 are disposed in the cold zone 14, and hence do not undergo decontamination or other biological safety procedures that are applicable to personnel and items disposed in the hot zone 10. The medical imaging instruments 16, 18 can, for example, be operated by personnel located in the cold zone 14 who are not wearing sealed environmental suits. However, in some cases one or more auxiliary instruments 30, 32 are disposed in the hot zone 10 and are configured to cooperate with the medical imaging instrument 16, 18 to image the subject disposed in the hot zone 10 through the imaging window 20. In the illustrated embodiment, the auxiliary instruments include a subject table 30 used to move the subject into the interior volume 22 to coincide with the imaging volume of one of the medical imaging instruments 16, 18, and a local radio frequency (RF) coil 32 such as may be used in conjunction with an MR scanner. Other devices such as electrocardiographic (EKG) monitors, respiratory monitors, SpO₂ monitors, thermometers, speakers, microphones, displays, cameras, monitors, workstation interfaces, heaters, automatic door drives, or so forth are also contemplated as auxiliary instruments.

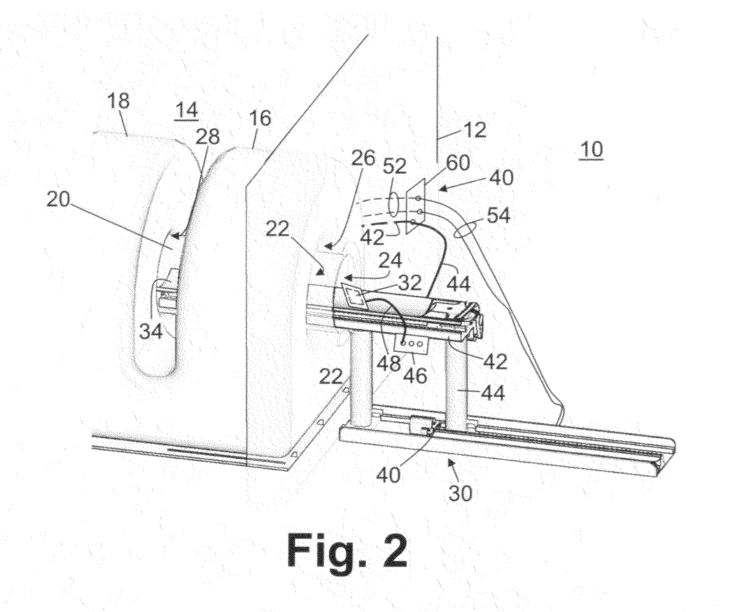

With reference to FIGS. 1 and 2, in FIG. 1 the subject table 30 is shown with a tabletop or pallet 34 fully withdrawn from the interior volume 22 of the generally hollow imaging window 20. Additionally, FIG. 1 shows the second medical imaging instrument 18 moved away from the first medical imaging instrument 16 by a distance D. In the illustrated embodiment, the second medical imaging instrument 18 is moved away on rails 36, so as to facilitate certain repairs or maintenance of the medical imaging instruments 16, 18. For example, if one of the medical imaging instruments 16, 18 is a CT scanner, separating the medical imaging instruments 16, 18 by the distance D may facilitate removal of a gantry housing panel of the CT scanner to access the x-ray tube (not shown) for replacement. FIG. 2 shows the isolation system with the second medical imaging instrument 18 moved adjacent the first medical imaging instrument 16 (that is, the separation distance D has been removed by moving the second medical imaging instrument along the rails 36 toward the first medical imaging instrument 16). Additionally, in FIG. 2 the tabletop or pallet 34 has been moved into the interior volume 22 of the generally hollow imaging window 20 and into alignment with the bore 22 of the first medical imaging instrument 16. In the illustrated embodiment, this insertion of the tabletop or pallet 34 is accomplished by a floor-mounted drive system 40 that moves an intermediate support 42 (including a rear pedestal 44) on which the tabletop or pallet 34 rests into the interior volume 22 of the generally hollow imaging window 20. Although not illustrated, in the example subject table 30, the tabletop or pallet 34 can be moved further into the interior volume 22 of the generally hollow imaging window 20 so as to align with the bore 28 of the second medical imaging instrument 18 through the mechanism of a second drive system (not shown) built into the intermediate support 42. The subject table 30 is an illustrative example, and other subject table configurations can be employed. Moreover, the subject table 30 and local RF coil 32 are illustrative examples of auxiliary instruments disposed in the hot zone 10, and other auxiliary instruments such as a set of electrocardiographic (EKG) leads, a respiratory monitor, or so forth can be disposed in the hot zone 10.

An electrical patch panel 40 is mounted on the barrier 12 to provide electrical interconnection between the medical imaging instruments 16, 18 and the auxiliary instruments 30, 32. Although not illustrated, the electrical patch panel 40 may provide ingress and egress of electrical power or signals for other purposes. Some example types of communication via the patch panel 40 may include, for example: transmission of a radio frequency excitation signal produced by an RF transmitter (not shown) in the cold zone 14 to the RF coil 32; transmission of a magnetic resonance signal from the RF coil 32 to an RF receiver (not shown) in the cold zone 14; transmission of electrical power and/or control signals from the cold zone 14 to the hot zone 10 for powering and/or controlling the subject table 30; transmission of EKG signals from EKG leads in the hot zone to an EKG monitor disposed in the cold zone 14 (EKG-related components not shown); a video or audio feed (not shown), and so forth.

In FIGS. 1 and 2, an example cold- or ambient-side cable 42 connects an MR scanner of the medical imaging instruments 16, 18 to a connector of the patch panel 40 while a corresponding hot- or isolation-side cable 44 continues from the patch panel connector to a connector of a user panel 46 disposed in the hot zone 10. A user cable 48 runs from the connector of the user panel 46 to the local RF coil 32, so that the combination of the cold-side and hot-side cables 42, 44, electrical patch panel 40, user panel 46, and user cable 48 effectuate connection of the local RF coil 32 disposed in the hot zone 10 and the MR scanner disposed in the cold zone 14. Similarly, example cold- or ambient-side cables 52 connect one of the medical imaging instruments 16, 18 to a connector of the patch panel 40 while corresponding hot- or isolation-side cables 54 continue from the patch panel connectors to the subject table 30, so that the combination of the cold-side and hot-side cables 52, 54 and the electrical patch panel 40 effectuate connection of the subject table 30 disposed in the hot zone 10 and the medical imaging instrument disposed in the cold zone 14.

The cold-side cables 42, 52 are disposed in the cold zone 14, and accordingly do not undergo the decontamination procedures employed in the hot zone 10. Accordingly, the cold-side cables 42, 52 can have insulation not designed to withstand corrosive substances used in decontamination in the hot zone 10. In contrast, the hot-side cables 44, 54 are disposed in the hot zone 10, and accordingly do undergo decontamination in accordance with the BSL-4 or other isolation standard employed in the hot zone 10. Accordingly, the hot-side cables 44, 54 have insulation designed to withstand corrosive substances or high temperatures used in decontamination in the hot zone 10. For example, the hot-side cables 44, 54 may include a polytetrafluorethylene (PTFE) insulation. In FIGS. 1 and 2, the difference between the cold-side cables 42, 52 and the hot-side cables 44, 54 is denoted by using dashed lines to illustrate the cold-side cables 42, 52 and solid lines to illustrate the hot-side cables 44, 54.

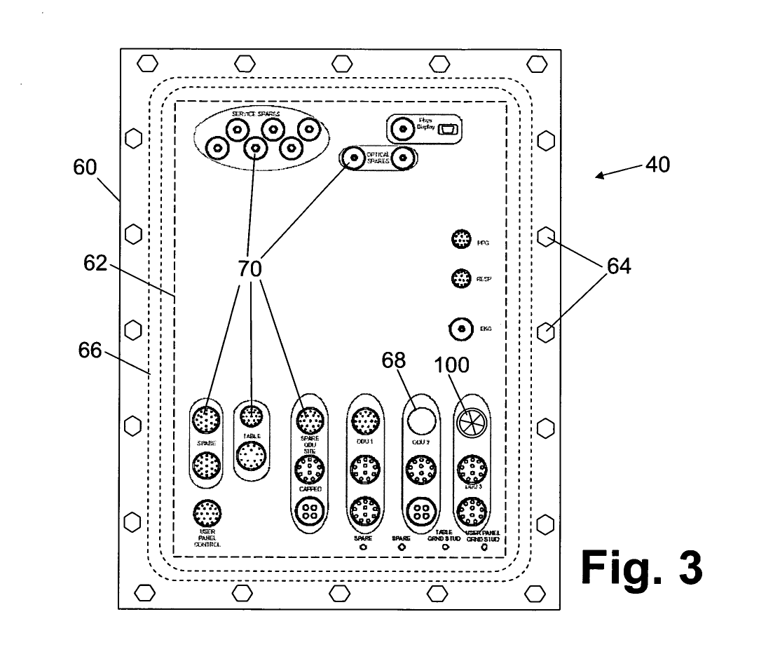

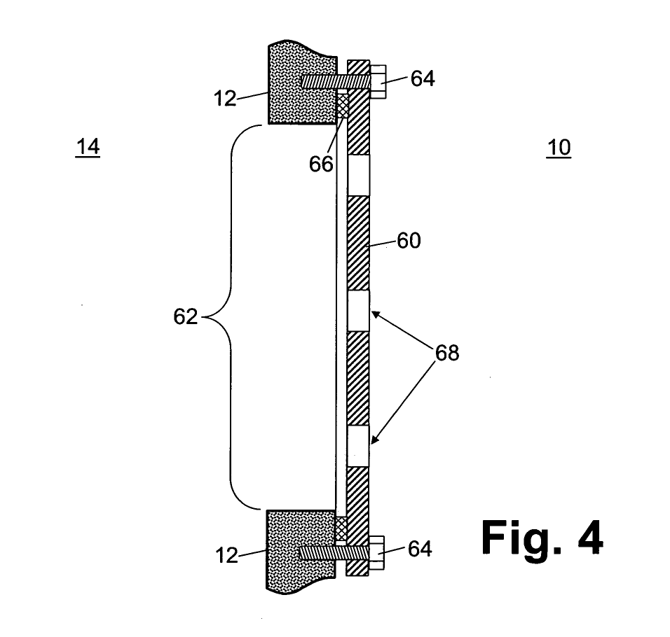

With continuing reference to FIGS. 1 and 2, and with further reference to FIGS. 3 and 4, the electrical patch panel 40 is further described. The patch panel 40 includes a through-hole panel 60 that is mounted aligned with an opening 62 (indicated in phantom in FIG. 3) in the barrier 12. Suitable fasteners 64 secure the through-hole panel 60 to the barrier 12. An annular gasket 66 (shown in phantom in FIG. 3), O-ring, or other seal is disposed between the through-hole panel 60 and the barrier 12 around the edge of the opening 62 to hermetically seal the opening 62 via the fastened through-hole panel 60. The through-hole panel 60 includes a plurality of through-holes 68 into which electrical feedthroughs 70 are inserted. In FIG. 3 a single through-hole 68 is shown without an inserted electrical feedthrough for illustrative purposes—however, in the completely assembled electrical patch panel 40 every through-hole 68 has an inserted electrical feedthrough or some other suitable plug to provide hermetic sealing of the through-hole. In FIG. 4, the electrical feedthroughs are not shown. The illustrated electrical patch panel 40 includes the through-hole panel 60 mounted to the barrier 12; however, it is also contemplated to integrate that through-hole panel with the barrier, for example by drilling through-holes directly into the barrier 12 to directly receive the electrical feedthroughs.

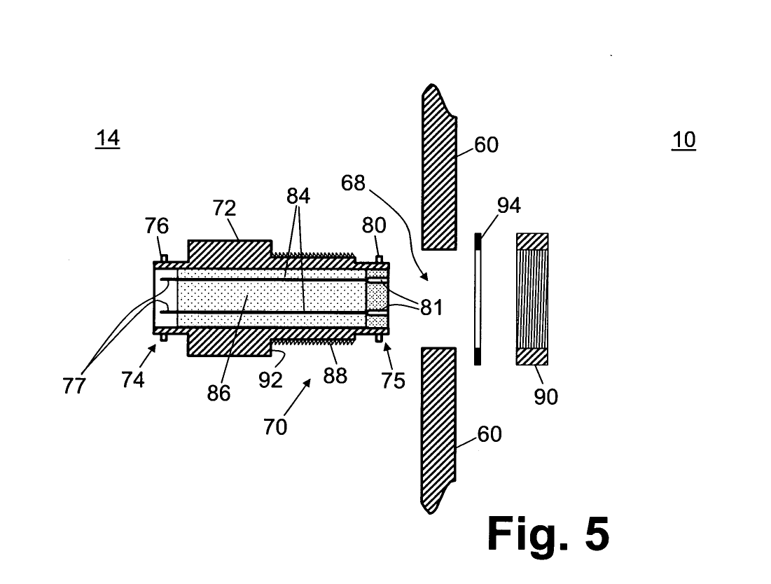

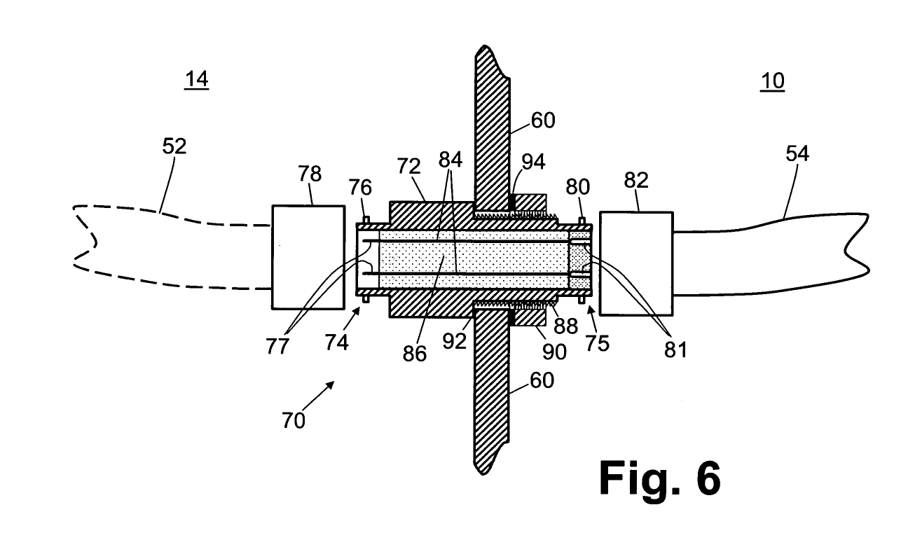

With reference to FIGS. 5 and 6, an example electrical feedthrough 70 that connects with one of the cold-side cables 52 and one of the hot-side cables 54 is further described. The electrical feedthrough 70 includes a housing 72 disposed in one of the through-holes 68 of the through-hole panel 60. A cold- or ambient-side electrical receptacle 74 extends from the housing 72 into the cold zone 14. A hot- or isolation-side electrical receptacle 75 extends from the housing 72 into the hot zone 10. In the illustrated embodiment, the cold-side receptacle 74 includes bayonet-style locking pins 76 and conductive pins 77 that fit into sockets (not shown) of a mating connector 78 of the cold-side cable 52, while the hot-side receptacle 75 includes bayonet-style locking pins 80 and conductive sockets 81 that receive pins (not shown) of a mating connector 82 of the hot-side cable 54. More generally, however, each of the cold-side and hot-side electrical receptacles may be either a female or male receptacle, and can take the form of a plug, socket, or so forth, and may use substantially any type of securing mechanism such as the illustrated bayonet-style locking pins, or a threaded mechanical connection, or a frictional securing connection, or so forth. One or more electrical conductors 84 are disposed in the housing 72 and electrically connect the conductive pins 77 of the cold-side electrical receptacle 74 and the conductive sockets 81 of the hot-side electrical receptacle 75. Potting material 86 is disposed in the housing 72 to pot the one or more electrical conductors 84 in the housing 72 and to isolate the hot-side electrical receptacle 75 from the cold- or ambient-side electrical receptacle 74. Moreover, although the illustrated conductors 84 are straight, the conductors can be twisted, bent, or otherwise shaped to accommodate different spatial conductive pin or conductive socket arrangements at the cold-side and hot-side electrical receptacles. In general, the conductive pins and/or conductive sockets of the hot-side and cold-side electrical receptacles can have any configuration.

A sealing fastener secures each electrical feedthrough 70 in its through-hole 68 and seals an interface or gap between an edge of the through-hole 68 and the electrical feedthrough 70. In the illustrated embodiment, the sealing fastener includes threading 88 on the housing 72 that mates with a threaded nut 90 disposed in the hot zone 10. Tightening the nut 90 onto the threads 88 pulls the nut 90 and a flange 92 of the housing 72 together such that the edges of the through-hole 68 are secured between the housing flange 92 and the nut 90. The illustrated sealing fastener also includes an annular sealing gasket 94 disposed between the edges of the through-hole 68 and the nut 90 to ensure hermetic sealing of the interface or gap between the edge of the through-hole 68 and the electrical feedthrough 70. The potting material 82 of the electrical feedthrough 70 and the sealing fastener 86, 88, 90, 92 cooperatively seal the opening of the through-hole 68 to isolate the hot zone 10 from the cold zone 14. The sealing fastener may optionally have other configurations and/or may include other components such as a washer or so forth.

In some embodiments, the electrical feedthrough 70 is based on a PotCon™ bulkhead connector available from Douglas Electrical Components, Inc. (Rockaway, N.J., USA). The PotCon™ connector is a bulkhead connector for porting electricity into and out of vacuum chambers, and includes a housing, conductors potted inside the housing with a low-outgassing epoxy sealant, electrical receptacles on the atmosphere and vacuum sides of the housing, and a nitrile rubber sealing gasket that provides a vacuum-tight seal. However, at least that portion of the electrical patch panel 40 which is exposed to the hot zone should be substantially resistant to one or more corrosive substances used in decontamination of the electrical patch panel 40. Typical corrosive substances used in decontamination complying with the BSL-4 isolation standard include Clydox-S, Microchem, Quat TB, Para-Formaldehyde, Chlorine-Dioxide, Vaporized Hydrogen Peroxide, and Ammonium Carbonate. Strong oxidants are typically effective corrosive substances for use in BSL-4 level decontamination. The nitrile rubber sealing gasket of the PotCon™ bulkhead connector is not substantially resistant to these corrosives. Accordingly, in some embodiments the PotCon™ bulkhead connector is used for the electrical feedthrough 70, but with the nitrile rubber sealing gasket replaced by an annular gasket of a more corrosive-resistant material such as polytetrafluorethylene (PTFE), which is substantially resistant to Clydox-S, Microchem, Quat TB, Para-Formaldehyde, Chlorine-Dioxide, Vaporized Hydrogen Peroxide, and Ammonium Carbonate. The sealing gasket 66 for sealing the through-hole panel 60 to the barrier 12 is also suitably made of PTFE. Other suitably corrosive-resistant materials besides PTFE can be used for the gaskets 66, 94 as well as for the insulation of the hot-side cables 44, 54.

Advantageously, the cold-side and hot-side electrical receptacles 74, 75 enables cables to be connected and disconnected from the patch panel 40 without breaking the containment seal of the hot zone 10. With reference back to FIG. 3, it is seen that in the example patch panel 40 the various electrical feedthroughs 70 are not all the same, but rather there are several different types of electrical feedthroughs 70 each having different numbers and/or configurations (e.g., spatial arrangements) of conductive pins or conductive sockets.

Moreover, it is straightforward to incorporate redundancy into the patch panel, by including extra electrical feedthroughs of the same type. Redundancy allows increased capacity to be added at a later date without breaking containment to add additional electrical feedthroughs. The hot-side electrical receptacle 75 of unused redundant feedthroughs are optionally capped by a cap, such as the example cap 100 shown in FIG. 3, to further reduce the likelihood that the contagion might escape via the unused redundant electrical feedthrough. Advantageously, there is no extraneous wiring extending away from such unused receptacles.

Although the electrical feedthroughs 70 promote connection and disconnection of cabling, it may be disadvantageous to make such connections and disconnections frequently at the patch panel 40. For example, the local RF coil 32 may be frequently connected and disconnected, for example to swap out a different local RF coil or local RF coil array, or to remove the local RF coil entirely when performing large-volume imaging employing a whole-body RF coil built into the MR scanner. Making such frequent connections and disconnections at the patch panel 40 may create the possibility of damaging or wearing out the electrical feedthrough, which could result in the electrical feedthrough being electrically non-functional and/or could generate a leak in the seal of the electrical feedthrough. In such cases, the user panel 46 shown in FIGS. 1 and 2 is convenient. As shown in these FIGURES, the hot-side cable 44 runs from the electrical feedthrough of the patch panel 40 to an electrical connection of the user panel 46. The user can then conveniently connect and disconnect the user cable 48 to and from the user panel 46 to effectuate connection and disconnection of the local RF coil 32, without unduly stressing the patch panel 40.

The illustrated patch panel 40 operatively electrically connects the one or more medical imaging instruments 16, 18 and the one or more auxiliary instruments 30, 32. However, it will be appreciated that the patch panel may also be used to provide ingress and/or egress of electrical power and/or electrical signals of substantially any type into and/or out of a hot zone of a biological, radioactive, or toxic chemical isolation system. The BSL-4 compliant hot zone 10 is an illustrative example, and patch panels such as the panel 40 illustrated herein may be used in conjunction with biological hot zones of other BSL levels, in conjunction with biological hot zones following other isolation standards besides the BSL level standards, in conjunction with nuclear hot zones, in conjunction with toxic chemical hot zones, and so forth.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

Having thus described the preferred embodiments, the invention is now claimed to be:

1. An electrical patch panel for use in communicating electrical power or electrical signals across a barrier between an isolation zone and an ambient zone, the patch panel comprising:
a through-hole panel mounted on the barrier between the isolation zone and the ambient zone; and
a plurality of electrical feedthroughs each including a housing disposed in a through-hole of the through-hole panel, an ambient-side electrical receptacle exposed to the ambient zone, an isolation-side electrical receptacle exposed to the isolation zone and electrically connected with the ambient-side electrical receptacle, and potting material disposed in the housing that isolates the isolation-side electrical receptacle from the ambient-side electrical receptacle, an interface or gap between an edge of the through-hole and the electrical feedthrough being sealed such that a pressure differential can be maintained between the isolation and ambient zones.

2. The electrical patch panel as set forth in claim 1, further including:
a sealing fastener securing each electrical feedthrough in its through-hole and sealing the interface or gap between the edge of the through-hole and the electrical feedthrough, the potting material of the electrical feedthrough and the sealing fastener cooperatively isolating the isolation zone from the ambient zone such that the pressure differential can be maintained between the isolation and ambient zones.

3. The electrical patch panel as set forth in claim 1, wherein the isolation zone is a hot zone maintained at BSL-4 isolation, the ambient zone is a cold zone not maintained at BSL-4 isolation, and at least that portion of the electrical patch panel which is exposed to the isolation zone is substantially resistant to a BSL-4 decontamination chemistry used in decontamination of the hot zone.

4. The electrical patch panel as set forth in claim 3, further including:
a hot-side electrical cable having a mating connector connected with the hot-side receptacle of a selected electrical feedthrough, the hot-side electrical cable having insulation that is substantially resistant to the BSL-4 decontamination chemistry; and
a cold-side electrical cable having a mating connector connected with the cold-side receptacle of the selected electrical feedthrough, the cold-side electrical cable and the hot-side electrical cable being electrically connected via the selected electrical feedthrough.

5. The electrical patch panel as set forth in claim 4, wherein the cold-side electrical cable is not substantially resistant to the BSL-4 decontamination chemistry.

6. The electrical patch panel as set forth in claim 3, further including:
an annular gasket disposed around the housing to seal the interface or gap between the edge of the through-hole and the electrical feedthrough.

7. The electrical patch panel as set forth in claim 6, wherein the annular gasket is a polytetrafluorethylene gasket.

8. The electrical patch panel as set forth in claim 1, wherein at least that portion of the electrical patch panel which is exposed to the isolation zone is resistant to biological decontamination chemicals.

9. The electrical patch panel as set forth in claim 1, wherein the potting material of each electrical feedthrough provides vacuum-tight isolation of the isolation-side electrical receptacle from the ambient-side electrical receptacle.

10. The electrical patch panel as set forth in claim 1, wherein the isolation environment complies with the BSL-4 isolation standard, and the potting material of each electrical feedthrough and the seal of the interface or gap between the edge of the through-hole and the electrical feedthrough provide isolation of the isolation zone from the ambient zone complying with the BSL-4 isolation standard.

11. The electrical patch panel as set forth in claim 1, wherein at least some of the electrical feedthroughs include an isolation-side electrical receptacle with a plurality of conductors electrically connected with corresponding conductors of the ambient-side electrical receptacle.

12. The electrical patch panel as set forth in claim 11, wherein the conductors of the isolation-side and ambient-side electrical receptacles are selected from a group consisting of conductive pins and conductive sockets.

13. The electrical patch panel as set forth in claim 1, wherein the plurality of electrical feedthroughs include a plurality of different types of isolation-side electrical receptacles, and further includes at least two of each type of isolation-side electrical receptacle.

14. A medical imaging system comprising:
a medical imaging instrument disposed in a cold zone and arranged to image a subject disposed in a hot zone; and
at least one electrical feedthrough including a housing sealed in a barrier between the hot zone and the cold zone, a cold-side electrical receptacle accessible from the cold zone, and a hot-side electrical receptacle accessible from the hot zone, the medical imaging instrument being electrically accessible from the hot zone via the at least one electrical feedthrough.

15. The medical imaging system as set forth in claim 14, further including:
an imaging window arranged at the barrier isolating the hot zone from the cold zone.

16. The medical imaging system as set forth in claim 15, wherein the imaging window is generally hollow and extends into the cold zone to define an interior volume having an opening communicating with the hot zone, the interior volume of the generally hollow imaging window being isolated from the cold zone.

17. The medical imaging system as set forth in claim 16, wherein the generally hollow imaging window extends into the cold zone such that the interior volume coincides with an imaging volume of the medical imaging instrument, the medical imaging system further including:
a subject table configured to extend into the interior volume of the generally hollow imaging window to place a subject disposed on the subject table into the imaging volume of the medical imaging instrument.

18. The medical imaging system as set forth in claim 17, wherein the medical imaging instrument includes at least one of a positron emission tomography scanner, a computed tomography scanner, a magnetic resonance scanner, and an x-ray imager.

19. The medical imaging system as set forth in claim 14, further including:
at least one auxiliary instrument disposed in the hot zone and electrically connected with the disposed in a cold zone via the at least one electrical feedthrough.

20. The medical imaging system as set forth in claim 19, wherein the medical imaging instrument is a magnetic resonance scanner, and the at least one auxiliary instrument includes:
one or more local radio frequency coils disposed in the hot zone and operatively electrically connected with the magnetic resonance scanner disposed in the cold zone via the at least one electrical feedthrough.

21. The medical imaging system as set forth in claim 14, wherein the barrier includes:
a through-hole panel including at least one through-hole in which the housing of the at least one electrical feedthrough is sealed.

22. The medical imaging system as set forth in claim 14, further including:
a user electrical panel disposed in the hot zone and connected by at least one hot-side electrical cable with the at least one electrical feedthrough; and
at least one user cable having a first end operatively connected with at least one instrument disposed in the hot zone and a second end detachably connectable with the user electrical panel.

23. The medical imaging system as set forth in claim 14, wherein the hot zone is isolated in compliance with the BSL-4 isolation standard.

24. A biological isolation system comprising:
a hot zone maintained at a selected level of biological isolation;
a through-hole panel mounted on a barrier between the hot zone and a cold zone that is not maintained at the selected level of biological isolation; and
a plurality of hermetically sealed electrical feedthroughs each including a housing, a cold-side electrical receptacle, and a hot-side electrical receptacle, the hermetically sealed electrical feedthroughs being hermetically sealed into through-holes of the through-hole panel with the hot-side electrical receptacle extending into the hot zone and the cold-side electrical receptacle extending into the cold zone, a surface of the through-hole panel exposed to the hot zone and a portion of the hermetically sealed electrical feedthroughs exposed to the hot zone being substantially resistant to one or more corrosive biological decontamination agents used in decontamination of the hot zone.

25. The biological isolation system as set forth in claim 24, wherein the hot zone is isolated to the BSL-4 level of biological isolation.

26. The biological isolation system as set forth in claim 24, wherein each hermetically sealed electrical feedthrough includes:
a potting material disposed in the housing and providing hermetic sealing isolating the hot-side and cold-side electrical receptacles from each other, the potting material not contributing to sealing of a gap or interface between the hermetically sealed electrical feedthrough and an edge of the through-hole.

27. The biological isolation system as set forth in claim 26, wherein each hermetically sealed electrical feedthrough further includes:
an annular gasket that hermetically seals the gap or interface between the hermetically sealed electrical feedthrough and an edge of the through-hole.

28. The biological isolation system as set forth in claim 27, wherein the annular gasket is resistant to strong oxidants.

29. The biological isolation system as set forth in claim 24, further including:
one or more medical imaging instruments disposed in the cold zone; and
a generally tubular imaging window having an interior volume communicating with the hot zone and isolated from the cold zone, the one or more medical imaging instruments arranged to image a volume coinciding with at least a portion of the interior volume of the imaging window.

30. The biological isolation system as set forth in claim 29, further including:
one or more auxiliary instruments disposed in the hot zone and operatively electrically communicating with the one or more medical imaging instruments disposed in the cold zone via the plurality of hermetically sealed electrical feedthroughs.

31. A method of providing electrical connections across a barrier of an isolation zone, the method comprising:
forming an opening in a barrier of an isolation zone;
inserting a sealed electrical feedthrough at the opening in the barrier; and
sealing an interface or gap between a housing of the sealed electrical feedthrough and an edge of the barrier.

32. The method as set forth in claim 31, further including:
electrical accessing an imaging system disposed outside the isolation zone via the sealed electrical feedthrough.

33. The method as set forth in claim 31, further including:
repeating the forming of an opening, the inserting and the sealing using two or more operatively identical sealed electrical feedthroughs to generate a corresponding two or more redundant electrical connections across the barrier.

34. The method as set forth in claim 33, further including:
installing a cap on an isolation-side electrical receptacle of an unused redundant sealed electrical feedthrough.

35. A biological containment environment for imaging comprising:
an isolation zone maintained at a selected level of biological isolation;
a medical imaging instrument disposed outside the isolation zone;
a tube extending from the isolation zone into an imaging region of the medical imaging instrument via which a subject in the isolation zone can be introduced into the imaging region without breaking containment of the isolation zone; and
a plurality of hermetically sealed electrical feedthroughs passing through a barrier delimiting the isolation zone, each hermetically sealed electrical feedthrough including a hermetically sealed housing with a cold-side electrical receptacle accessible from outside the isolation zone and a hot-side electrical receptacle accessible from within the isolation zone, the hermetically sealed electrical feedthroughs providing electrical communication between the isolation zone and the medical imaging instrument.

36. The biological containment environment as set forth in claim 35, wherein the tube is one of cylindrical and tapered and has one of a circular, elliptical, square, or rectangular cross-section.

37. The biological containment environment as set forth in claim 35, further comprising:
a panel sealed with an opening in the barrier, the plurality of hermetically sealed electrical feedthroughs sealed with and passing through the panel.
